(19) United States
(12) Patent Application Publication
(10) Pub. No.: US 2003/0039619 A1
(43) Pub. Date: Feb. 27, 2003

(54) Galenic formulation

(76) Inventors: Joachim Bunger, Gross-Umstadt (DE); Jutta zur Lage, Darmstadt (DE); Alexandra Axt, Rossdorf (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/182,122

(22) PCT Filed: Jan. 15, 2001
(86) PCT No.: PCT/EP01/00396

(30) Foreign Application Priority Data
Jan. 28, 2000 (DE) 10003786.0

(51) Int. Cl. A61K 31/522; A61K 31/353; A61K 7/42; A61K 31/12; A61K 31/15

(52) U.S. Cl. 424/59; 514/263.31; 514/456; 514/683; 514/640

(57) ABSTRACT

Cosmetic or dermatological formulations which comprise one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine and in addition

(a) one or more antioxidants

and/or

(b) one or more UV filters,

are distinguished, in particular, by the fact that the active ingredients present in the formulation according to the invention are stabilised.

The invention relates to stabilised cosmetic or dermatological formulations comprising auxiliaries and/or excipients and one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine.

The use of phylloquinone (vitamin K1) in cosmetic compositions is known.

Formulations comprising phylloquinone can be used, for example, for lightening the skin, for removing blood spots, small blood vessels and blue spots, against dark eye rings, for strengthening capillaries, for tightening the skin and/or for preventing skin irritation.

JP 053200039 discloses, for example, skin-lightening cosmetics which comprise vitamin K1.

EP 0 521 647 describes, for example, cosmetic compositions which comprise vitamin K1 and are suitable, in particular, for use on the skin areas around the eyes.

The use of 2-hydroxy-5-methyllaurophenone oxime, which is also known as HMLO or F5, in cosmetic compositions is disclosed, for example, in DE 41 16 123. The compositions described therein are suitable for the treatment of skin diseases that are accompanied by inflammation. The areas of application of these compositions are the pharmaceutical industry, human and veterinary medicine and cosmetics. In particular, it is described that the formulations comprising 2-hydroxy-5-methyllaurophenone oxime can be used, for example, for the therapy of psoriasis, various forms of eczema, irritative and toxic dermatitis, UV dermatitis and further allergic and/or inflammatory diseases of the skin and the integumentary appendages.

Vitamin A and vitamin E and their derivatives act, for example, as antiageing substances.

Vitamin H (biotin) can be used, for example, for the prophylaxis or treatment of skin ageing processes or of dry skin states.

Allantoin and bisabolol function, for example, as antiinflammatory active ingredients.

Nicotinic acid derivatives and caffeine serve, for example, as active ingredients which stimulate blood flow.

However, active ingredients which are to be incorporated into cosmetic or dermatological formulations are often unstable and can be damaged in the formulation. The damage can be caused, for example, by reaction with atmospheric oxygen or by the absorption of UV rays. The molecules damaged in this way can, for example, lose their colour and/or their effectiveness through their structural change.

For example, it has hitherto only been possible with difficulty to formulate substances such as vitamin K1 in a stable manner in a cosmetic or dermatological formulation. After the incorporation of vitamin K1 into cosmetic or dermatological formulations, these formulations change, for example, their colour through exposure to UV light and to an increased extent through exposure to UV light and the simultaneous presence of atmospheric oxygen. This results in a heterogenous brown coloration of the originally pale, for example pale-yellow, cosmetic or dermatological formulation. Colour changes of this type are perceived to be unaesthetic by the customers.

For the above-mentioned reasons, it is necessary to stabilise active ingredients in cosmetic or dermatological formulations.

The object was therefore to provide cosmetic and dermatological formulations comprising auxiliaries and/or excipients and one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine which are distinguished by the fact that the active ingredients have the highest possible stability in the formulation, and the formulations are, in addition, also stabilised against discoloration.

Surprisingly, it has now been found that this object is achieved by the provision of cosmetic and dermatological formulations comprising auxiliaries and/or excipients and one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine if they additionally comprise

(a) one or more antioxidants

and/or

(b) one or more UV filters.

The invention thus relates to cosmetic or dermatological formulations comprising auxiliaries and/or excipients and one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine, characterised in that they additionally comprise

(a) one or more antioxidants

and/or

(b) one or more UV filters.

The invention furthermore relates to the use of one or more compounds selected from antioxidants and UV filters for the protection and/or stabilisation of active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine present in a cosmetic or dermatological formulation.

The invention furthermore relates to the use of one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine and one or more compounds selected from antioxidants and UV filters for the preparation of a cosmetic or dermatological formulation. the invention also relates to the use of the said active ingredients, antioxidants and UV filters for the preparation of a cosmetic or dermatological formulation for the protection or care of human or animal skin.

The invention furthermore relates to a process for the preparation of a cosmetic or dermatological formulation, characterised in that one or more active ingredients selected from phylloquinone, 2-hydroxy-5-methyl-laurophenone oxime, vitamin A and its derivatives, vitamin E and its derivatives, vitamin H, allantoin, bisabolol, nicotinic acid derivatives and caffeine and one or more substances selected from antioxidants and UV filters, if desired with auxiliaries and/or excipients, are converted into a suitable cosmetic or dermatological formulation form.

The derivatives of vitamin A are preferably selected from vitamin A propionate, vitamin A palmitate and vitamin A acetate.

The derivatives of vitamin E are preferably selected from DL-α-tocopherol, tocopherol E acetate and tocopherol hydrogensuccinate.

The preferred nicotinic acid derivative is nicotinamide.

The antioxidants and/or UV filters present in the cosmetic or dermatological formulations according to the invention stabilise the active ingredients likewise present. This has, for example, an advantageous effect in the form of an increase in the storage stability of the formulations according to the invention.

The cosmetic or dermatological formulations according to the invention can be employed in the area of skin protection/skin care. They can be employed for the protection or care of both human and animal skin. Protection of human skin is preferred.

Preferred active ingredients which are present in the formulations according to the invention are preferably selected from phylloquinone and 2-hydroxy-5-methyllaurophenone oxime.

The formulations according to the invention may comprise the antioxidants known from the specialist literature, for example flavonoids, coumaranones, amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), and coniferyl benzoate of benzoin resin, rutinic acid and derivatives thereof, α-glycosyl rutin, ferulic acid, furfurylidineglucitol, carnosine, butylhydroxytoluene (BHT), butylhydroxyanisole, nordihydroguaiaretic acid, trihydroxy-butyrophenone, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the formulations according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

In a preferred embodiment of the invention, the cosmetic or dermatological formulation according to the invention comprises butylhydroxytoluene as antioxidant.

In a further preferred embodiment, the cosmetic or dermatological formulation according to the invention comprises one or more compounds selected from flavonoids and/or coumaranones as antioxidant.

Flavonoids are taken to mean the glycosides of flavonones, flavones, 3-hydroxyflavones (=flavonols), aurones, isoflavones and rotenoids [Römpp Chemie Lexikon [Römpp's Lexicon of Chemistry], Volume 9, 1993]. For the purposes of the present invention, however, they are also taken to mean the aglycones, i.e. the sugar-free constituents, and the derivatives of flavonoids and aglycones. For the purposes of the present invention, the term coumaranones is also taken to mean the derivatives thereof.

Preferred flavonoids are derived from flavonones, flavones, 3-hydroxyflavones, aurones and isoflavones, in particular from flavonones, flavones, 3-hydroxyflavones and aurones.

The flavonones are characterised by the following basic structure:

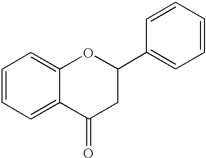

The flavones are characterised by the following basic structure:

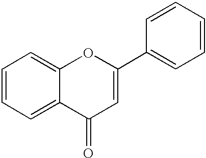

The 3-hydroxyflavones (flavonols) are characterised by the following basic structure:

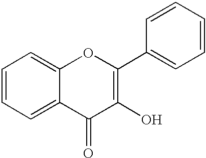

The isoflavones are characterised by the following basic structure:

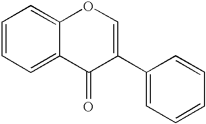

The aurones are characterised by the following basic structure:

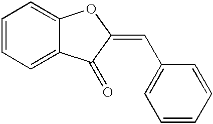

The coumaranones are characterised by the following basic structure:

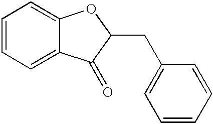

The flavonoids and coumaranones are preferably selected from the compounds of the formula (I):

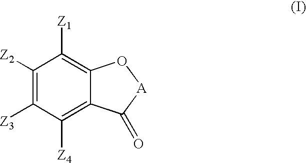

in which

Z₁ to Z₄ are each, independently of one another, H, OH, alkoxy, hydroxyalkoxy, mono- or oligoglycoside radicals, where the alkoxy and hydroxyalkoxy groups may be branched or unbranched and may have from 1 to 18 carbon atoms, and where sulfate or phosphate may also be bonded to the hydroxyl groups of the said radicals,

A is selected from the group consisting of the sub-formulae (IA), (IB) and (IC)

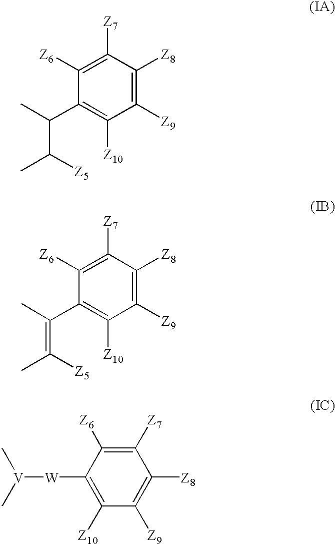

Z₅ is H, OH or OR,

R is a mono- or oligoglycoside radical,

Z₆ to Z₁₀ are as defined for the radicals Z₁ , to Z₄, and

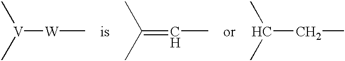

The alkoxy groups are preferably linear and have from 1 to 12 and preferably from 1 to 8 carbon atoms. These groups thus conform to the formula —O—(CH₂)ₘ—H, where m is 1, 2, 3, 4, 5, 6, 7 or 8 and in particular from 1 to 5.

The hydroxyalkoxy groups are preferably linear and have from 2 to 12 and preferably from 2 to 8 carbon atoms. These groups thus conform to the formula —O—(CH₂)ₙ—OH, where n is 2, 3, 4, 5, 6, 7 or 8, in particular from 2 to 5 and extremely preferably 2.

The mono- and oligoglycoside radicals are preferably built up from 1 to 3 glycoside units. These units are preferably selected from the group consisting of the hexosyl radicals, in particular the rhamnosyl radicals and glucosyl radicals. However, other hexosyl radicals, for example allosyl, altrosyl, galactosyl, gulosyl, idosyl, mannosyl and talosyl, can also, if desired, advantageously be used. It may also be advantageous in accordance with the invention to use pentosyl radicals.

In a preferred embodiment,

Z₁ and Z₃ are H,

Z₂ and Z₄ are other than H, in particular are OH, methoxy, ethoxy or 2-hydroxyethoxy,

Z₅ is H, OH or a glycoside radical which is built up from 1 to 3, preferably from 1 or 2, glycoside units,

Z₆, Z₉ and Z₁₀ are H, and

Z₇ and Z₈ are other than H, in particular are OH, methoxy, ethoxy or 2-hydroxyethoxy.

In a further preferred embodiment, in particular if the water solubility of the flavonoids and coumaranones is to be increased, a sulfate or phosphate group is bonded to the hydroxyl groups. Suitable counterions are, for example, the ions of the alkali or alkaline earth metals, where these are selected, for example, from sodium or potassium.

In a further preferred embodiment, the flavonoids are selected from the following compounds: 4,6,3′,4′-tetrahydroxyaurone, quercetin, rutin, isoquercetin, anthocyanidin (cyanidin), eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin) and their sulfates and phosphates.

Of the flavonoids, particular preference is given to rutin and troxerutin. Very particular preference is given to troxerutin.

Of the coumaranones, preference is given to 4,6,3′,4′-tetrahydroxybenzylcoumaran-3-one.

Suitable organic UV filters are all UVA and UVB filters known to the person skilled in the art. For both UV ranges, there are many proven substances known from the specialist literature, for example

benzylidenecamphor derivatives, such as

3-(4′-methylbenzylidene)-dl-camphor (for example Eusolex ® 6300),

3-benzylidenecamphor (for example Mexoryl® SD),

polymers of N-{(2 and 4)-[(2-oxoborn-3-ylidene)methyl]benzyl}acryl-amide (for example Mexoryl® SW),

N,N,N-trimethyl-4-(2-oxoborn-3-ylidenemethyl)anilinium methylsulfate (for example Mexoryl® SK) or

α-(2-oxoborn-3-ylidene)toluene-4-sulfonic acid (for example Mexoryl® SL),

benzoyl- or dibenzoylmethanes, such as

1-(4-tert-butylphenyl)-3-(4-methoxyphenyl)propane-1,3-dione (for example Eusolex® 9020) or

4-isopropyidibenzoylmethane (for example Eusolex® 8020),

benzophenones, such as

2-hydroxy-4-methoxybenzophenone (for example Eusolex® 4360) or

2-hydroxy-4-methoxybenzophenone-5-sulfonic acid and its sodium salt (for example Uvinul® MS-40),

methoxycinnamic acid esters, such as

octyl methoxycinnamate (for example Eusolex® 2292),

isopentyl 4-methoxycinnamate, for example as a mixture of the isomers (for example Neo Heliopan® E 1000),

salicylate derivatives, such as

2-ethylhexyl salicylate (for example Eusolex® OS),

4-isopropylbenzyl salicylate (for example Megasol®) or

3,3,5-trimethylcyclohexyl salicylate (for example Eusolex® HMS),

4-aminobenzoic acid and derivatives, such as

4-aminobenzoic acid,

2-ethylhexyl 4-(dimethylamino)benzoate (for example Eusolex® 6007),

ethoxylated ethyl 4-aminobenzoate (for example Uvinul® P25),

and further substances, such as

2-ethylhexyl 2-cyano-3,3-diphenylacrylate (for example Eusolex® OCR),

2-phenylbenzimidazole-5-sulfonic acid and its potassium, sodium and triethanolamine salts (for example Eusolex® 232),

3,3′-(1,4-phenylenedimethylene)bis(7,7-dimethyl-2-oxobicyclo[2.2.1]-hept-1-ylmethanesulfonic acid and its salts (for example Mexoryl® SX) and

2,4,6-trianilino-(p-carbo-2′-ethylhexyl-1′-oxy)-1,3,5-triazine (for example Uvinul® T 150).

These organic UV filters are generally incorporated into the formulations according to the invention in an amount of from 0.5 to 10 per cent by weight, preferably 1-8%.

Further suitable organic UV filters are, for example,

2-(2H-benzotriazol-2-yl)-4-methyl-6-(2-methyl-3-( 1,3,3,3-tetramethyl-1-(trimethylsilyloxy)disiloxanyl)propyl)phenol (for example Silatrizole®),

2-ethylhexyl 4,4′-[(6-[4-((1 ,1 -dimethylethyl)aminocarbonyl)phenyl-amino]-1,3,5-triazine-2,4-diyl)diimino]bis(benzoate) (for example Uvasorb® HEB),

α-(trimethylsilyl)-ω-[trimethylsilyl)oxy]poly[oxy(dimethyl [and approximately 6% of methyl[2-[p-[2,2-bis(ethoxycarbonyl]vinyl]-phenoxy]-1-methyleneethyl] and approximately 1.5% of methyl[3-[p-[2,2-bis(ethoxycarbonyl)vinyl]phenoxy]propenyl] and from 0.1 to 0.4% of (methylhydrogen]silylene]] (n≈60) (for example Parsol® SLX) 2,2′-methylenebis(6-(2H-benzotriazol-2-yl)-4-( 1,1,3,3-tetramethylbutyl)-phenol) (for example Tinosorb® M)

2,2′-(1,4-phenylene)bis(1H-benzimidazole-4,6-disulfonic acid, mono-sodium salt) (Neo Heliopan® AP) and

2,4-bis{[4-(2-ethylhexyloxy)-2-hydroxy]phenyl}-6-(4-methoxyphenyl)-1,3,5-triazine (for example Tinosorb® S).

These organic UV filters are generally incorporated into the formulations according to the invention in an amount of from 0.5 to 20 per cent by weight, preferably 1-15%.

Conceivable inorganic UV filters are those from the group consisting of titanium dioxides, such as, for example, coated titanium dioxide (for example Eusolex® T-2000 or Eusolex® T-AQUA), zinc oxides (for example Sachtotec®), iron oxides and also cerium oxides. These inorganic UV filters are generally incorporated into the formulations according to the invention in an amount of from 0.5 to 20 per cent by weight, preferably 2-10%.

Preferred UV filters are zinc oxide, titanium oxide, 3-(4′-methylbenzylidine)-dl-camphor, 1-(4-tert-butylphenyl)-3-(4-methoxyphenyl)propane-1,3-dione, 4-isopropyidibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and its potassium, sodium and triethanolamine salts.

Particularly preferred UV filters are zinc oxide and titanium dioxide.

Of the formulations according to the invention comprising titanium dioxide, preference is given to those which, besides titanium dioxide, additionally comprise one or more further UV filters selected from 3-(4′-methylbenzylidene)-dl-camphor, 1-(4-tert-butylphenyl)-3-(4-methoxy-phenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethyihexyl 2-cyano-3,3-diphenylacrylate, 2-phenylbenzimidazole-5-sulfonic acid and their potassium, sodium and triethanolamine salts.

Of these formulations, particular preference is given to those which, besides titanium dioxide, additionally comprise the UV filters 2-hydroxy-4-methoxybenzophenone and/or octyl methoxycinnamate.

The formulation according to the invention is prepared by converting one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine and one or more substances selected from antioxidants and UV filters, if desired with auxiliaries and/or excipients, into a suitable formulation form. The auxiliaries and excipients originate from the group consisting of the vehicles, preservatives and other conventional auxiliaries.

The cosmetic or dermatological formulations according to the invention are applied externally.

Examples which may be mentioned of application forms of the formulations according to the invention are: solutions, suspensions, emulsions, pastes, ointments, gels, creams, lotions, powders, soaps, surfactant-containing cleansing preparations, oils and sprays. Examples of other application forms are sticks, shampoos and shower preparations. Any desired customary excipients, auxiliaries and, if desired, further active ingredients may be added to the formulation.

Preferred auxiliaries originate from the group consisting of preservatives, antioxidants, stabilisers, solubilisers, vitamins, colorants, odour improvers film formers, thickeners and humectants.

Ointments, pastes, creams and gels may comprise the customary excipients, for example animal and vegetable fats, waxes, paraffins, starch, tragacanth, cellulose derivatives, polyethylene glycols, silicones, bentonites, silica, talc and zinc oxide, or mixtures of these substances. Powders and sprays may comprise the customary excipients, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary excipients, such as solvents, solubility promoters and emulsifiers, for example water, ethanol, isopropanol, ethyl carbonate, ethyl acetate, benzyl alcohol, benzyl benzoate, propylene glycol, 1,3-butylglycol, oils, in particular cottonseed oil, peanut oil, wheatgerm oil, olive oil, castor oil and sesame oil, glycerol fatty acid esters, polyethylene glycols and fatty acid esters of sorbitan or mixtures of these substances.

Suspensions may comprise the customary excipients, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar agar and tragacanth, or mixtures of these substances.

Soaps may comprise the customary excipients, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products may comprise the customary excipients, such as salts of fatty alcohol sulfates, fatty alcohol ether sulfates, sulfosuccinic monoesters, fatty acid protein hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters, or mixtures of these substances. Face and body oils may comprise the customary excipients, such as synthetic oils, such as fatty acid esters, fatty alcohols, silicone oils, natural oils, such as vegetable oils and oily plant extracts, paraffin oils or lanolin oils, or mixtures of these substances.

Further typical cosmetic application forms are also lipsticks, lipcare sticks, mascara, eyeliner, eyeshadow, rouge, powder make-up, emulsion make-up and wax make-up, and sunscreen, presun and aftersun preparations.

In a particularly preferred embodiment of the invention, the cosmetic or dermatological formulations are in the form of a W/O emulsion. This applies in particular, for example, if phylloquinone is present as active ingredient in the cosmetic in the dermatological formulation.

The proportion of the one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine in the formulation according to the invention is preferably from 0.001 to 20% by weight, particularly preferably from 0.1 to 3% by weight, based on the entire formulation according to the invention.

The proportion of the one or more antioxidants in the formulation according to the invention is preferably from 0.001 to 5% by weight, particularly preferably from 0.01 to 2% by weight, based on the entire formulation according to the invention.

The proportion of the one or more UV filters in the formulation according to the invention is preferably from 0.05 to 20% by weight, particularly preferably from 1 to 10% by weight, based on the entire formulation according to the invention.

The stabilising action of the antioxidants and UV filters on the active ingredients in the formulations according to the invention can be determined by methods which are known to the person skilled in the art. For example, a formulation according to the invention and a corresponding formulation without antioxidants and UV filters can be stored under identical conditions (influence of light and air), and the stabilising effect of the antioxidants and UV filters can be determined by comparing the discoloration of these formulations.

All compounds or components which can be used in the formulations according to the invention are either known and commercially available or can be synthesised by known methods.

### EXAMPLES

The following examples serve to illustrate the invention and should not be regarded as a limitation. All % data are per cent by weight.

The INCI names of the raw materials used in the examples are indicated below in the original English terminology. They are as follows:

| Raw material | INCI |
|---|---|
| Antaron V-220 | PVP/Eicosene Glycol |
| Arlacel 165V | Glyceryl Stearate (and) PEG-100 |
| | Stearate |
| Arlacel 1689 | Sorbitol, Glycerol Esters |
| BHT | BHT |
| Beeswax | Beeswax |
| Cetiol | Oleyl Oleate |
| Cetiol V | Decyl Oleate |
| Cetyl alcohol | Cetyl Alcohol |
| Cutina HR | Hydrogenated Castor Oil |
| Emulsifier E2155 | Stearyl Alcohol (and) Steareth-7 (and) |
| | Steareth-10 |
| Eusolex 2292 | Octyl Methoxycinnamate |
| Eusolex 4360 | Benzophenone-3 |
| Eusolex 6300 | 4-Methylbenzylidene Campher |
| Eusolex 9020 | Butyl Methoxydibenzoylmethane |
| EusolexT-2000 | Titanium Dioxide, Alumina; Simethicone |
| Eusolex T-AQUA | Aqua, Titanium Dioxide, Alumina, |
| | Sodium Methaphosphate, Phenoxy- |
| | ethanol, Sodium Methylparaben |
| Euxyl K100 | Benzyl Alcohol, Methylchloroisothia- |
| | zoline, Methylisothiazoline |
| Germaben II-E | Propylene Glycol, Diazolidinyl Urea, |
| | Methylparabene, Propylparabene |
| Glycerin | Glycerin |
| Glycerol | Glycerin |
| Imwitor 900 | Glyceryl Stearate |
| Isolan PDI | Diisosteaoryl Polyglyceryl-3- |
| | Diisostearate |
| Isopropyl myristate | Isopropyl Myristate |
| Isopropyl palmitate | Isopropyl Palmitate |
| Karion F liquid | Sorbitol |
| Lanette 18 | Stearyl Alcohol |
| Lanette O | Cetearyl Alcohol |
| Lanolin | Lanolin |
| Lunacera M | Microwax |
| Luvitol EHO | Cetearyl Octanoate |
| Magnesium sulfate | Magnesium Sulfate |
| Miglyol 812 | Caprylic/Capric Triglyceride |
| Miglyol 812 neutral oil | Caprylic/Capric Triglyceride |
| Mirasil CM | Cyclomethicone |
| Mirasil DM 350 | Dimethicone |
| Oxynex K liquid | PEG-8, Tocopherol, Ascorbyl Palmi- |
| | tate, Ascorbic Acid, Citric Acid |
| Paraffin | Mineral Oil |
| Paraffin, liquid | Mineral Oil |
| Propane-1,2-diol | Propylene Glycol |
| Rutin | Rutin |
| Tegin M | Glyceryl Stearate |
| Teginacid H | Glyceryl Stearate (and) Ceteth-20 |
| Tego Care 450 | Polyglyceryl-3 Methylglucose Distearate |
| Titriplex III | Disodium EDTA |
| Trihydroxyethylrutin | Troxerutin |
| Vitamin K1 | Phytonadione |

### Examples 1-3

### BHT as Antioxidant

### Example 1

O/W emulsions according to the invention comprising vitamin K1 are prepared from the following components:

| | | | | % by wt. |
|---|---|---|---|---|
| A | Paraffin | (Art. No. 107160) | (1) | 8.0 |
| | Isopropyl myristate | (Art. No. 822102) | (1) | 7.0 |
| | Lanette O | | (2) | 3.0 |
| | Arlacel 165V | | (3) | 5.0 |
| | BHT | | (4) | x |
| B | Water, demineralised | | | to 100 |
| | Glycerol, 87% | (Art. No. 104091) | (1) | 3.0 |
| | Preservatives | | | q.s. |
| C | Vitamin K1 | (Art. No. 501890) | (1) | 1.0 |

In Example 1A x=0.05, and in Example 1B x=0.1.

Preservatives which can be used are

0.05% of propyl 4-hydroxybenzoate (Art. No.130173) or

0.15% of methyl 4-hydroxybenzoate (Art. No.130174)

Preparation:

Phases A and B are warmed separately to 75° C., and phase A is subsequently introduced into phase B with stirring. The combined phases are homogenised. The mixture is then cooled with stirring, and phase C is added at 30° C.

Properties:

The emulsions have a pale yellow colour and a pH of 5.7.

Sources of supply:

(1) Merck KGaA

(2) Henkel KGaA

(3) Uniqema

(4) Rhodia

The emulsions of Examples 1A and 1B are stored for one month in air under daylight. The comparison used is an analogous O/W emulsion, but comprising no BHT. The creams of Examples 1A and 1B exhibit significantly less discoloration after this time than the emulsion without BHT. The emulsions of Examples 1A and 1B exhibit no difference in discoloration.

### Example 2

A W/O emulsion according to the invention comprising vitamin K1 is prepared from the following components:

| | | | | % by wt. |
|---|---|---|---|---|
| A | Arlacel 1689 | | (3) | 6.0 |
| | Paraffin, liquid | (Art. No. 107162) | (1) | 17.0 |
| | Isopropyl myristate | (Art. No. 822102) | (1) | 5.0 |
| | BHT | | (4) | 0.05 |
| B | Water, demineralised | | | to 100 |
| | Glycerol, 87% | (Art. No. 104091) | (1) | 4.0 |
| | Magnesium sulfate | (Art. No. 105886) | (1) | 0.5 |
| | Preservatives | | | q.s. |
| C | Vitamin K1 | (Art. No. 501890) | (1) | 1.0 |

Preservatives which can be used are

0.05% of propyl 4-hydroxybenzoate (Art. No. 130173) or

0.15% of methyl 4-hydroxybenzoate (Art. No. 130174)

Preparation:

Phases A and B are warmed separately to 75° C., and phase B is subsequently introduced into phase A with stirring. The combined phases are homogenised. The mixture is then cooled with stirring, and phase C is added at 30° C.

Properties:

The emulsion has a pale yellow colour.

Sources of supply:

(1) Merck KGaA

(2) Henkel KGaA

(3) Uniqema

(4) Rhodia

The emulsion of Example 2 is tested analogously to those of Examples 1A and 1B. The emulsion of Example 2 exhibits significantly less discoloration than the emulsion of Example 1A.

### Example 3

A W/O emulsion according to the invention comprising vitamin K1 is prepared from the following components:

| | | | | % by wt. |
|---|---|---|---|---|
| A | Isolan PDI | | (3) | 3.0 |
| | Paraffin, liquid | | (1) | 17.0 |
| | Isopropyl myristate | (Art. No. 822102) | (1) | 5.0 |
| | Beeswax | (Art. No. 111544) | (1) | 0.2 |
| | Cutina HR | | (2) | 0.3 |
| | BHT | | (4) | 0.05 |
| B | Water, demineralised | | | to 100 |
| | Glycerol, 87% | (Art. No. 104091) | (1) | 4.0 |
| | Magnesium sulfate | | (1) | 1.0 |
| | Preservatives | | | q.s. |
| C | Vitamin K1 | (Art. No. 501890) | (1) | 1.0 |

Preservatives which can be used are

0.05% of propyl 4-hydroxybenzoate (Art. No. 130173) or

0.15% of methyl 4-hydroxybenzoate (Art. No. 130174)

Preparation:

Phases A and B are warmed separately to 75° C., and phase B is subsequently introduced into phase A with stirring. The combined phases are homogenised. The mixture is then cooled with stirring, and phase C is added at 30° C.

Properties:

The emulsion has a pale yellow colour.

Sources of supply:

(1) Merck KGaA

(2) Henkel KGaA

(3) Uniqema

(4) Rhodia

The emulsion of Example 3 is tested analogously to those of Examples 1A and 1B. The emulsion of Example 3 exhibits significantly less discoloration than the emulsion of Example 1A. The discoloration of the emulsion of Example 3 is slightly more pronounced than the discoloration of the emulsion of Example 2.

### Examples 4-5

### Bioflavonoids as Antioxidants

### Example 4

O/W emulsions according to the invention comprising vitamin K1 are prepared from the following components:

| | | | | % by wt. |
|---|---|---|---|---|
| A | Paraffin | (Art. No. 107160) | (1) | 8.0 |
| | Isopropyl myristate | (Art. No. 822102) | (1) | 7.0 |
| | Lanette O | | (2) | 3.0 |
| | Arlacel 165V | | (3) | 5.0 |
| B | Water, demineralised | | | to 100 |
| | Glycerol, 87% | (Art. No. 104091) | (1) | 3.0 |
| | Preservatives | | | q.s. |
| | Trihydroxyethylrutin | (Art. No. 509002) | (1) | x |
| C | Vitamin K1 | (Art. No. 501890) | (1) | 1.0 |

In Example 4A x=0.05, in Example 4B x=0.1, in Example 4C x=0.5, and in Example 4D x=1.0.

Preservatives which can be used are

0.05% of propyl 4-hydroxybenzoate (Art. No. 130173) or

0.15% of methyl 4-hydroxybenzoate (Art. No. 130174)

Preparation:

Phases A and B are warmed separately to 75° C., and phase A is subsequently introduced into phase B with stirring. The combined phases are homogenised. The mixture is then cooled with stirring, and phase C is added at 30° C.

Sources of supply:

(1) Merck KGaA

(2) Henkel KGaA

(3) Uniqema

The emulsions of Example 4 are tested analogously to those of Examples 1A and 1B. The emulsions of Example 4 exhibit significantly less discoloration than the emulsion of Example 1A. In addition, it is noted that the discoloration becomes less with increasing concentration of trihydroxyethylrutin and the emulsions comprising 0.5% by weight and 1.0% by weight of trihydroxyethylrutin are the least discoloured. The emulsions of Examples 4C and 4D (0.5% by weight and 1.0% by weight of trihydroxyethylrutin) exhibit no differences in discoloration.

### Example 5

O/W emulsions according to the invention comprising vitamin K1 are prepared from the following components:

| | | | | % by wt. |
|---|---|---|---|---|
| A | Tego Care 450 | | (5) | 3.0 |
| | Tegin M | | (5) | 1.0 |
| | Lanette 18 | | (2) | 1.0 |
| | Miglyol 812 | | (2) | 7.0 |
| | Mirasil DM 350 | | (4) | 0.5 |
| | Mirasil CM | | (4) | 2.0 |
| | Isopropyl palmitate | | (1) | 5.0 |
| | BHT | | (4) | x |
| | Rutin | (Art. No. 500017) | (1) | y |
| B | Water, demineralised | | | to 100 |
| | Glycerol, 87% | (Art. No. 104091) | (1) | 3.0 |
| | Preservatives | | | q.s. |
| | Trihydroxyethylrutin | (Art. No. 509002) | (1) | z |
| C | Vitamin K1 | (Art. No. 501890) | (1) | 1.0 |

In Example 5A x=0.05, y =0 and z=0;

in Example 5B x=0, y=0 and z=0.1;

in Example 5C x=0, y=0 and z=0.5;

in Example 5D x=0, y=0.1 and z=0 and

in Example 5E x=0, y=0.5 and z=0.

Preservatives which can be used are

0.05% of propyl 4-hydroxybenzoate (Art. No. 130173) or

0.15% of methyl 4-hydroxybenzoate (Art. No. 130174)

Preparation:

Phases A and B are warmed separately to 70° C., and phase A is subsequently introduced into phase B with stirring. The combined phases are homogenised. The mixture is then cooled with stirring, and phase C is added at 30° C.

Sources of supply:

(1) Merck KGaA

(2) Henkel KGaA

(3) Uniqema

(4) Rhodia

(5) Th. Goldschmidt AG

The emulsions of Example 5 are tested analogously to those of Examples 1A and 1B. The emulsions of Examples 5B and 5C (trihydroxyethylrutin as antioxidant) exhibit the least change in colour, then the emulsions of Examples 5D and 5E (rutin as antioxidant) and finally the emulsion of Example 5A by comparison exhibits the greatest change in colour.

### Examples 6-10

### Formulations Comprising UV Filters

### Example 6

An O/W emulsion according to the invention comprising vitamin K1 is prepared from the following components:

| | | | | % by wt. |
|---|---|---|---|---|
| A | Arlacel 165V | | (2) | 10.0 |
| | Paraffin, liquid | (Art. No. 1.07162) | (1) | 25.0 |
| | Cetyl alcohol | (Art. No. 1.00989) | (1) | 2.0 |
| | Lanolin | | (3) | 2.0 |
| | Oxynex K liquid | (Art. No. 1.08324) | (1) | 0.05 |
| B | Zinc oxide | (Art. No. 1.30148) | (1) | 10.0 |
| | Karion F liquid | (Art. No. 1.02993) | (1) | 3.0 |
| | Glycerin | (Art. No. 1.04093) | (1) | 2.0 |
| | Titriplex III | (Art. No. 1.08421) | (1) | 0.05 |
| | Germaben II-E | | (4) | 0.5 |
| | Water, demineralised | | | to 100 |
| C | Vitamin K1 | | (1) | 1.0 |

Preparation:

Phase A is warmed to 75° C. and phase B separately to 80° C., and subsequently phase B is introduced slowly into phase A with stirring. The combined phases are homogenised. The mixture is then cooled with stirring, and phase C is added at 35° C.

Sources of supply:

(1) Merck KGaA

(2) ICI

(3) Henry Lamotte

(4) ISP

The zinc oxide significantly counters discoloration of the emulsion due to storage under daylight.

### Example 7

O/W emulsions according to the invention comprising vitamin K1 are prepared from the following components:

| | | | | % by wt. |
|---|---|---|---|---|
| A | Eusolex T-2000 | (Art. No. 105373) | (1) | a |
| | Eusolex 4360 | (Art. No. 105376) | (1) | b |
| | Emulsifier E2155 | | (2) | 2.0 |
| | Teginacid H | | (2) | 2.0 |
| | Luvitol EHO | | (3) | 6.0 |
| | Imwitor 900 | | (4) | 3.0 |
| | Cetiol | | (5) | 5.0 |
| | Lunacera M | | (6) | 1.0 |
| | Miglyol 812 neutral oil | | (4) | c |
| | Antaron V-220 | | (7) | 1.0 |
| B | Propane-1,2-diol | (Art. No. 107478) | (1) | 4.0 |
| | Water, demineralised | | | to 100 |
| | Germaben II-E | | (7) | 0.5 |
| C | Vitamin K1 | | (1) | 1.0 |

In Example 7A a=6.0, b=3.0 and c=3.0

In Example 7B a=6.0, b=0 and c=6.0

In Example 7C a=0, b=3.0 and c=3.0

Preparation:

Phase A is warmed to 75° C. and phase B separately to 80° C., and subsequently phase B is introduced slowly into phase A with stirring. The combined phases are homogenised. The mixture is then cooled with stirring, and phase C is added at 35° C.

Sources of supply:

(1) Merck KGaA

(2) Th. Goldschmidt

(3) BASF AG

(4) Hüls Troisdorf AG

(5) Henkel KGaA

(6) H. B. Fuller GmbH

(7) ISP

The discoloration of the emulsions by the following influences is investigated qualitatively below. Here, for example, “emulsion 1<emulsion 2” means that emulsion 1 is discoloured significantly less than emulsion 2. “<<” means that this effect is particularly pronounced.

Storage under the influence of daylight (4 weeks): emulsion 7A<emulsion 7B<emulsion 7C

Exposure to UV light (15 min.; ‘Suntest CPS’ instrument, 80 W/m²): emulsion 7A<emulsion 7C<<emulsion 7B

Storage in the absence of light (4 weeks): emulsion 7B<emulsion 7A<emulsion 7C

The combination of the UV filters Eusolex T-2000 and Eusolex 4360 counters discoloration of the emulsion significantly more in real terms than the said UV filters alone.

### Example 8

O/W emulsions according to the invention comprising vitamin K1 are prepared from the following components:

| | | | | % by wt. |
|---|---|---|---|---|
| A | Eusolex 2292 | (Art. No. 1.05382) | (1) | a |
| | Eusolex 4360 | (Art. No. 1.05376) | (1) | b |
| | Emulsifier E2155 | | (2) | 3.0 |
| | Teginacid H | | (2) | 3.0 |
| | Luvitol EHO | | (3) | c |
| | Imwitor 900 | | (4) | 3.0 |
| | Cetiol | | (5) | 4.0 |
| | Lunacera M | | (6) | 1.0 |
| | Miglyol 812 neutral oil | | (4) | 3.0 |
| B | Eusolex T-AQUA | | (1) | d |
| | Propane-1,2-diol | (Art. No. 1.07478) | (1) | 4.0 |
| | Allantoin | | (1) | 0.2 |
| | Germaben II-E | | (7) | 0.5 |
| | Water, demineralised | | | to 100 |
| C | Vitamin K1 | | (1) | 1.0 |

In Example 8A a=7.0, b=3.0, c=10.5 and d=16.7

In Example 8B a=0, b=0, c=20.5 and d=16.7

In Example 8C a=7.0, b=3.0, c=13.5 and d=0

Preparation:

Phase A is warmed to 75° C. and phase B separately to 80° C., and subsequently phase B is introduced slowly into phase A with stirring. The combined phases are homogenised. The mixture is then cooled with stirring, and phase C is added at 35° C.

Sources of supply:

(1) Merck KGaA

(2) Th. Goldschmidt

(3) BASF AG

(4) Hüls Troisdorf AG

(5) Henkel KGaA

(6) H. B. Fuller GmbH

(7) ISP

The discoloration of the emulsions by the following influences is investigated qualitatively below. Here, for example, “emulsion 1<emulsion 2” means that emulsion 1 is discoloured significantly less than emulsion 2. “<<” means that this effect is particularly pronounced. “Emulsion 1˜emulsion 2” means that emulsion 1 is discoloured by the corresponding influence in a similar manner to emulsion 2.

Storage under the influence of daylight (4 weeks): emulsion 8A<emulsion 8B<<emulsion 8C

Exposure to UV light (15 min.; ‘Suntest CPS’ instrument, 80 W/m²): emulsion 8A<<emulsion 8C<emulsion 8B

Storage in the absence of light (4 weeks): emulsion 8A˜emulsion 8B<<emulsion 8C

All the said UV filter combinations exhibit a significant improvement in colour stability. The best results are achieved with titanium dioxide or, after UV irradiation, with titanium dioxide in the simultaneous presence of Eusolex 2292 and Eusolex 4360.

### Example 9

A W/O emulsion according to the invention comprising vitamin K1 is prepared from the following components:

| | | | | % by wt. |
|---|---|---|---|---|
| A | Eusolex 2292 | (Art. No. 1.05382) | (1) | 7.0 |
| | Eusolex 9020 | (Art. No. 1.05844) | (1) | 2.0 |
| | Isolan PDI | | (2) | 3.0 |
| | Cetiol V | | (3) | 6.0 |
| | Luvitol EHO | | (4) | 6.5 |
| | Lunacera M | | (5) | 0.5 |
| B | Glycerin (about 87%) | (Art. No. 1.04091) | (1) | 3.0 |
| | Magnesium sulfate | | | |
| | heptahydrate | (Art. No. 1.05882) | (1) | 1.0 |
| | Germaben II-E | | (6) | 0.5 |
| | Water, demineralised | | | to 100 |
| C | Vitamin K1 | | (1) | 1.0 |

Preparation:

Phase A is warmed to 75° C. and phase B separately to 80° C., and subsequently phase B is introduced slowly into phase A with stirring. The combined phases are homogenised. The mixture is then cooled with stirring, and phase C is added at 35° C.

Sources of Supply:

(1) Merck KGaA

(2) Th. Goldschmidt AG

(3) Henkel KGaA

(4) BASF AG

(5) H. B. Fuller GmbH

(6) ISP

The combination of the light-protection filters Eusolex 2292 and Eusolex 9020 significantly counters discoloration of the emulsion due to storage under daylight, due to UV irradiation or due to storage in the absence of light.

### Example 10

A W/O emulsion according to the invention comprising vitamin K1 is prepared from the following components:

| | | | | % by wt. |
|---|---|---|---|---|
| A | Eusolex T-2000 | (Art. No. 1.05373) | (1) | 3.0 |
| | Eusolex 4360 | (Art. No. 1.05376) | (1) | 2.0 |
| | Isolan PDI | | (2) | 3.0 |
| | Cetiol V | | (3) | 8.0 |
| | Luvitol EHO | | (4) | 8.5 |
| | Lunacera M | | (5) | 0.5 |
| B | Glycerin (about 87%) | (Art. No. 1.04091) | (1) | 3.0 |
| | Magnesium sulfate | | | |
| | heptahydrate | (Art. No. 1.05882) | (1) | 1.0 |
| | Euxyl K100 | | (6) | 0.5 |
| | Water, demineralised | | | to 100 |
| C | Vitamin K1 | | (1) | 1.0 |

Preparation:

Phase A is warmed to 75° C. and phase B separately to 80° C., and subsequently phase B is introduced slowly into phase A with stirring. The combined phases are homogenised. The mixture is then cooled with stirring, and phase C is added at 35° C.

Sources of supply:

(1) Merck KGaA

(2) Th. Goldschmidt AG

(3) Henkel KGaA

(4) BASF AG

(5) H. B. Fuller GmbH

(6) Schülke & Mayr

This emulsion exhibits significantly less discoloration compared with the emulsions investigated in Examples 6-9.

1. Cosmetic or dermatological formulation comprising auxiliaries and/or excipients and one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine, characterised in that
(a) one or more antioxidants
and/or
(b) one or more UV filters
are additionally present.

2. Cosmetic or dermatological formulation according to claim 1, characterised in that the active ingredient present is phylloquinone and/or 2-hydroxy-5-methyllaurophenone oxime.

3. Cosmetic or dermatological formulation according to one of claims 1 and 2, characterised in that the antioxidant present is butylhydroxytoluene.

4. Cosmetic or dermatological formulation according to one of claims 1 and 2, characterised in that the antioxidant present is one or more compounds selected from flavonoids and/or coumaranones.

5. Cosmetic or dermatological formulation according to claim 4, characterised in that the flavonoids and coumaranones are selected from the compounds of the formula (I): 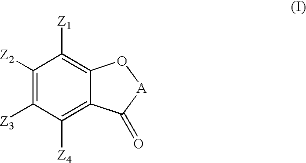
in which
Z₁ to Z₄ are each, independently of one another, H, OH, alkoxy, hydroxyalkoxy, mono- or oligoglycoside radicals, where the alkoxy and hydroxyalkoxy groups may be branched or unbranched and may have from 1 to 18 carbon atoms, and where sulfate or phosphate may also be bonded to the hydroxyl groups of the said radicals,
A is selected from the group consisting of the sub-formulae (IA), (IB) and (IC) 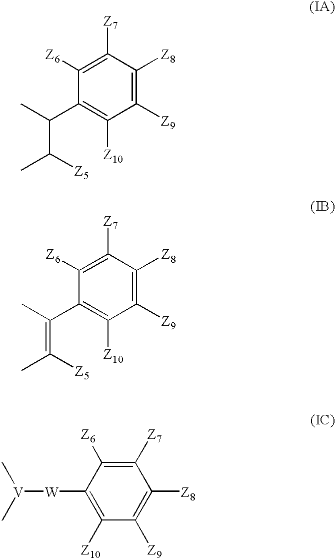
Z₅ is H, OH or OR,
R is a mono- or oligoglycoside radical,
Z₆ to Z₁₀ are as defined for the radicals Z₁ to Z₄, and

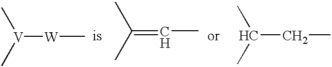

6. Cosmetic or dermatological formulation according to claim 5, characterised in that the flavonoid is rutin or troxerutin.

7. Cosmetic or dermatological formulation according to one of claims 1 to 6, characterised in that the one or more UV filters are selected from the following substances: zinc oxide, titanium dioxide, 3-(4′-methylbenzylldene)-dl-camphor, 1-(4-tert-butylphenyl)-3-(4-methoxy-phenyl)propane-1,3-dione, 4-isopropyidibenzoylmethane, 2-hydroxy-4-methoxybenzophenone, octyl methoxycinnamate, 3,3,5-trimethyl-cyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and its potassium, sodium and triethanolamine salts.

8. Cosmetic or dermatological formulation according to claim 7, characterised in that the UV filter present is zinc oxide and/or titanium dioxide.

9. Cosmetic or dermatological formulation according to claim 8, characterised in that the UV filter present is titanium dioxide and one or more further UV filters selected from 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzo-phenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and its potassium, sodium and triethanolamine salts.

10. Cosmetic or dermatological formulation according to claim 9, characterised in that the further UV filters are selected from 2-hydroxy-4-methoxybenzophenone and octyl methoxycinnamate.

11. Cosmetic or dermatological formulation according to one of claims 1 to 10, characterised in that the proportion of the active ingredients is from 0.001 to 20% by weight, the proportion of the one or more antioxidants is from 0.001 to 5% by weight, and the proportion of the one or more UV filters is from 0.05 to 20% by weight, in each case based on the entire formulation.

12. Cosmetic or dermatological formulation according to one of claims 1 to 11, characterised in that it is in the form of a solution, a suspension, an emulsion, a paste, an ointment, a gel, a cream, a lotion, a powder, a soap, a surfactant-containing cleansing preparation, an oil, a lipstick, a lip-care stick, a mascara, an eyeliner, eyeshadow, rouge, powder, emulsion or wax make-up, a sunscreen, pre-sun or after-sun preparation or a spray, a stick, a shampoo or a shower preparation.

13. Cosmetic or dermatological formulation according to claim 12, characterised in that it is in the form of a W/O emulsion.

14. Use of one or more compounds selected from antioxidants and UV filters for the protection and/or stabilisation of active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine present in a cosmetic or dermatological formulation.

15. Use of one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine and one or more substances selected from antioxidants and/or UV filters for the preparation of a cosmetic or dermatological formulation.

16. Use according to claim 15 for the protection or care of human or animal skin.

17. Process for the preparation of a cosmetic or dermatological formulation, characterised in that one or more active ingredients selected from phylloquinone, 2-hydroxy-5-methyllaurophenone oxime, vitamin A and its derivatives, vitamin E and its derivatives, vitamin H, allantoin, bisabolol, nicotinic acid derivatives and caffeine and one or more substances selected from antioxidants and UV filters, if desired with auxiliaries and/or excipients, are converted into a suitable cosmetic or dermatological formulation form.
